(19) United States
(12) Patent Application Publication
(10) Pub. No.: US 2009/0136898 A1
(43) Pub. Date: May 28, 2009

(54) Tap Drill for Dental Implant

(76) Inventor: Soo Hong KIM, Busan (KR)

Correspondence Address: PARK LAW FIRM, 3255 WILSHIRE BLVD, SUITE 1110, LOS ANGELES, CA, 90010, US

(21) Appl. No.: 12/030,139

(22) Filed: Feb. 12, 2008

(30) Foreign Application Priority Data
Nov. 26, 2007 (KR) 10-2007-0120580

(51) Int. Cl. A61C 1/08; A61B 17/16

(52) U.S. Cl. 433/165

(57) ABSTRACT

A tap drill for dental implant for forming a space for coupling a fixture of the implant in an alveolar bone, includes: a shank part coupled with a torque transferring mechanism; and a tap part connected with the shank part, the tap part having: a tapping screw thread formed on the outer peripheral surface thereof for forming a space to insert and fix a fixture of the dental implant thereto; and cutting blades radially formed from the center of a front end face thereof, which is perpendicular to a rotary axis of the tap drill, whereby the tap drill can simultaneously perform a tapping work, on the outer peripheral surface of the tap part, against a cartilaginous tissue or an alveolar bone of a patient's body, and a cutting work on the front end face of the tap part while the tap part is rotated by the torque transferring mechanism.

### BACKGROUND OF THE INVENTION

1. Field of the Invention

The present invention relates to a tap drill for a dental implant, and more particularly, to a tap drill for a dental implant, which has a tapping screw thread formed on the outer peripheral surface thereof and a cutting blade formed on a front end face thereof so that an operator can perform a tapping work on a cartilaginous tissue or an alveolar bone of a patient' body and a cutting work on the cartilaginous tissue or the alveolar bone in a front end direction thereof to thereby enhance functions of the tap drill, which can promote simplification in an dental implant surgery since the tapping work and the cutting work can be performed simultaneously, and which can precisely and stably crush materials located on the front end of the tap drill while being stably supported and rotated at a low speed by the tapping screw thread to thereby enhance precision and stability in the cutting work.

2. Background Art

A dental implant is an artificial dental root, which is implanted in an alveolar bone in stead of a damaged dental root removed from the alveolar bone. In general, the dental implant includes a fixture implanted and coupled to the alveolar bone, and prosthesis, such as an abutment, coupled to the fixture to fix an artificial crown. Such a dental implant is implanted in the alveolar bone using coupling and assembling instruments of various kinds.

Typically, a dental implant surgery is performed by using an implant surgical kit. The implant surgical kit includes: various drills for forming a basic hole, such as a point drill and a tab drill; various drivers adapted to allow an operator to use various parts and drills of an implant system using handling instruments, such as a hand piece and a dental ratchet; and various gauges for measurement during the implant surgery.

In relation with such an implant surgical kit for the dental implant, Korean Patent No. 0,453,726 discloses a “fixture driver structure for implant surgery”, Korean Patent No. 0,453,728 discloses a “fixture driver structure for implant surgery”, Korean Utility Model Registration No. 0,300,718 discloses a “torque wrench for implant surgery”, and Korean Patent No. 0,539,414 discloses a “drill set for dental surgery”, which are invented by the same inventor as the present invention.

Here, in order to completely implant the fixture of the dental implant into the alveolar bone of a patient's body, the implant surgery is performed through the steps of: boring the basic hole in the alveolar bone using the point drill; expanding the basic hole (namely, expanding the basic hole while changing diameters of the drills ranging from a smaller one to a larger one) using general drills for cutting to form a fixture insertion hole; processing the fixture insertion hole in such a way as to be provided a female-screwed inner peripheral surface thereof; and fixing a male-screwed fixture to the alveolar bone through coupling between the male-screwed fixture and the female-screwed fixture insertion hole.

As described above, the tap drill according to the prior art is only used for processing the inner peripheral surface of the fixture insertion hole into the female-screwed inner peripheral surface since it simply has a screw-type tapping thread formed on the outer peripheral surface thereof. Accordingly, the tap drill is restricted in its function.

Furthermore, in connection with drills used for a general implant surgery, Korean Patent No. 10-0619145 discloses an “implant drill for sinus lift technique”, Korean Patent Laid-open No. 10-2004-0032216 discloses a “drill for implant surgery”, Korean Utility Model Registration No. 20-0323647 discloses a “drill for implant surgery”, and Korean Utility Model Registration No. 20-0365546 discloses “counter drill and tap drill”. Such drills related with the implant surgery according to the prior arts are used to simply bore a hole, cut the surface of a bone, or form a space for implanting the fixture of the implant. So, the drills according to the above-mentioned prior arts are also restricted in the original function thereof.

Meanwhile, the artificial tooth can occupy the proper place and perform the function of the patient' permanent tooth only when the fixture of the implant must be implanted stably and firmly. Hence, in case where a width or thickness is smaller than a width or thickness to implant the implant due to a shortage of the remaining bone in the alveolar bone, a surgery for supplementing the shortage is performed.

Particularly, in case where a molar tooth is pulled out from the alveolar bone, since absorption of the alveolar bone is progressed widely as time goes, in many cases, a dental surgery for supplementing the shortage of the alveolar bone is performed before the implant surgery is performed in relation with the molar teeth part.

For the dental surgery to supplement the shortage of the remaining bone of the alveolar bone, now, there are a sinus lift technique, a ridge split, a block bone grafting, and so on.

The sinus lift technique is a method including the steps of: lifting a maxillary sinus mucosa formed above the maxilla, on which the molar teeth part is located, and adapted to ventilate air with the nose so as to form a space; performing a bone grafting in the formed space; and implanting the implant in the bone-grafted space. Moreover, the ridge split is a method including the steps of: boring a hole in the center of the alveolar bone, which is narrowed as a long time passes in a state where a tooth is lost; widening the hole to the right and left to form a space; and implanting the implant in the formed space.

Furthermore, the block bone grafting is a method including the steps of: extracting a bone in a block type from a cartilaginous tissue of the patient's body, such as a ramus part of the mandibule or a hipbone; and fixing the extracted block-type bone on a portion, where the implant will be implanted, to create an alveolar bone.

In more detail, the sinus lift technique is a method to perform a bone grafting through the steps of: boring a hole ranging from the alveolar bone, on which the molar teeth part is located, to the maxilla; crushing a compact bone below the maxillary sinus mucosa by tapping the maxilla several times using an osteotome and a mallet without any damage of the maxillary sinus mucosa so as to form a fine hole of a diameter ranging from 2 mm to 3 mm; lifting the maxillary sinus mucosa by putting crushed bone fragments into the fine hole; and continuously putting the crushed bone fragments into a space formed below the lifted maxillary sinus mucosa. Since the bone grafting is impossible when the crushed bone fragments are introduced into the maxillary sinus, it is the most important to prevent a damage of the maxillary sinus mucosa during the dental surgery. However, since the conventional sinus lift technique is a method to crush the compact bone of the maxilla by tapping the osteotome of a chisel type with the mallet, the technique requires a highly-advanced and experienced skill. Accordingly, a development of a new type tool, which can crush the compact bone in safer, is needed.

In connection with the above need, Korean Patent No. 10-0,630,304 discloses a “reamer for a dental implant”. The reamer for the dental implant includes: a cutting part for forming a hole to implant an implant into a bone; and a connection part extending downwardly from the cutting part and having a diameter smaller than that of the cutting part, wherein the cutting part includes: a protruding face formed in such a way as to upheave a face, including the outer periphery, of the front face of the upper end of the cutting part so as to form a stepped jaw; a depressed face formed in such a way that a face of the front face of the cutting part is depressed on the contrary to the protruding face, the depressed face having a reverse inclination in a counterclockwise direction; a cutting edge horizontally formed at a connection portion between the stepped jaw and the protruding face, the cutting edge evenly dispersing power in a horizontal line contact with the maxillary sinus mucosa in order to allow an operator to perform a cutting work in safe without any damage of the maxillary sinus mucosa; and a discharge path formed by cutting a face of the upper end of the cutting part, namely, ranging from a predetermined portion between the protruding face and the depressed face to a portion slightly higher than the lower end of the cutting part.

However, the reamer for the dental implant is restricted in its function since it is a method to crush the compact bone below the maxillary sinus mucosa after boring the hole from the alveolar bone to the maxilla.

### SUMMARY OF THE INVENTION

Accordingly, the present invention has been made to solve the above-mentioned problems occurring in the prior arts, and it is an object of the present invention to provide a new-type tap drill for a dental implant, which has a tapping screw thread formed on the outer peripheral surface thereof and a cutting blade formed at a front end face thereof so as to perform a tapping work on a cartilaginous tissue or an alveolar bone of a patient' body and a cutting work on the cartilaginous tissue or the alveolar bone in a front end direction thereof, thereby enhancing functions of the tap drill.

It is another object of the present invention to provide a new-type tap drill for a dental implant, which can simultaneously perform the tapping work for inserting and fixing a fixture of the implant and a cutting work required in a dental surgery, such as the sinus lift technique or the ridge split for a stable implantation of the fixture, to stably implant the fixture in the alveolar bone, thereby promoting simplification in the implant surgery.

It is a further object of the present invention to provide a new-type tap drill for a dental implant, which can precisely and stably crush materials existing on the front end of the tap drill while being stably supported and rotated at a low speed by the tapping screw thread, thereby enhancing precision and stability in the cutting work.

It is a still further object of the present invention to provide a new-type tap drill for a dental implant, which can crush a compact bone below the maxillary sinus mucosa while being stably supported and rotated at a low speed by the tapping screw thread when it is applied to the sinus lift technique, thereby stably crushing the compact bone without any damage of the maxillary sinus mucosa.

To accomplish the above object, according to the present invention, there is provided a tap drill for a dental implant comprising: a shank part coupled with a torque transferring mechanism; and a tap part connected with the shank part, the tap part having: a tapping screw thread formed on the outer peripheral surface thereof for forming a space to insert and fix a fixture of the dental implant thereto; and cutting blades radially formed from the center of a front end face thereof, which is perpendicular to a rotary axis of the tap drill, whereby the tap drill can simultaneously perform a tapping work, on the outer peripheral surface of the tap part, against a cartilaginous tissue or an alveolar bone of a patient's body, and a cutting work on the front end face of the tap part while the tap part is rotated by the torque transferring mechanism.

In addition, the tap part adopts one of a straight type and a tapered type.

Furthermore, the shank part includes a coupling protrusion formed at an end thereof in such a way as to be coupled to a handpiece and an adapter mounting portion formed at the other end thereof in such a way as to be coupled to an adapter of a dental ratchet, so that the tap part is rotated by one selected from the handpiece and the dental ratchet.

Moreover, the adapter mounting portion includes a locking face taperedly formed on the outer peripheral surface of the shank part, and a contact face formed by processing the outer peripheral surface of the shank part in a polygonal shape.

According to the present invention, the tap drill for the dental implant can allow the operator to perform the tapping work against the cartilaginous tissue or the alveolar bone of the patient' body and the cutting work on the cartilaginous tissue or the alveolar bone in the front end direction thereof to thereby enhance functions of the tap drill, since the tap drill has the tapping screw thread formed on the outer peripheral surface thereof and the cutting blade formed at the front end face thereof.

Furthermore, the tap drill for the dental implant can simultaneously perform the tapping work for inserting and fixing a fixture of the implant and a cutting work required in a dental surgery, such as the sinus lift technique or the ridge split, to stably implant the fixture in the alveolar bone, thereby promoting simplification in the dental implant surgery.

Moreover, the tap drill for the dental implant can precisely and stably crush materials existing on the front end of the tap drill while being stably supported and rotated at a low speed by the tapping screw thread, thereby enhancing precision and stability in the cutting work when the operator performs the cutting work at the front end portion of the tap drill according to the present invention.

In addition, the tap drill for the dental implant can crush a compact bone below the maxillary sinus mucosa while being stably supported and rotated at a low speed by the tapping screw thread when it is applied to the sinus lift technique, thereby stably crushing the compact bone without any damage of the maxillary sinus mucosa.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a view for showing a technical idea of a tap drill for a dental implant according to the present invention;

FIG. 2(a) is a perspective view of a straight-type tap drill for a dental implant according to a first preferred embodiment of the present invention;

FIG. 2(b) is a sectional view of the straight-type tap drill for the dental implant;

FIG. 2(c) is a front view of a cutting blade mounted on the front end face of the straight-type tap drill for the dental implant;

FIG. 3(a) is a perspective view of a tapered-type tap drill for a dental implant according to a second preferred embodiment of the present invention;

FIG. 3(b) is a sectional view of the tapered-type tap drill for the dental implant;

FIG. 3(c) is a front view of a cutting blade mounted on the front end face of the tapered-type tap drill for the dental implant;

FIG. 4 is a view showing a state where a handpiece is coupled to the tap drill for the dental implant according to the present invention;

FIG. 5 is a view showing a state where a dental ratchet is coupled to the tap drill for the dental implant according to the present invention;

FIG. 6 is a view showing a used example of the straight-type tap drill for the dental implant according to the present invention; and

FIG. 7 is a view showing a used example of the tapered-type tap drill for the dental implant according to the present invention.

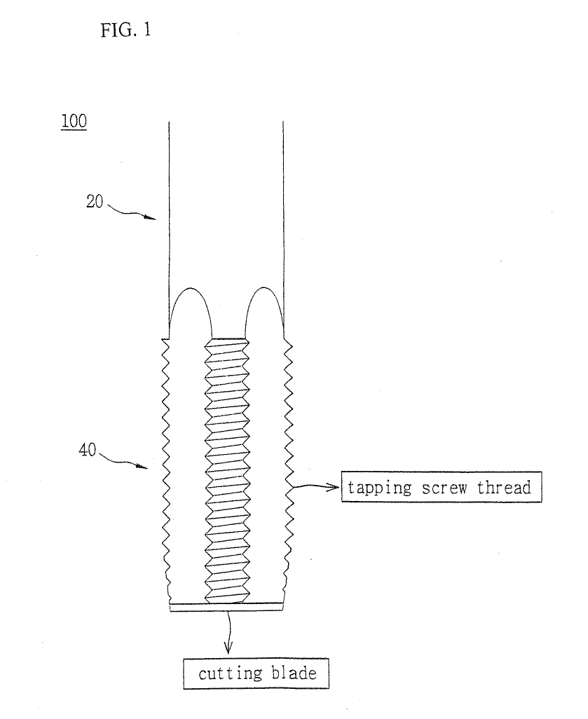

### DETAILED DESCRIPTION OF THE PREFERRED EMBODIMENT

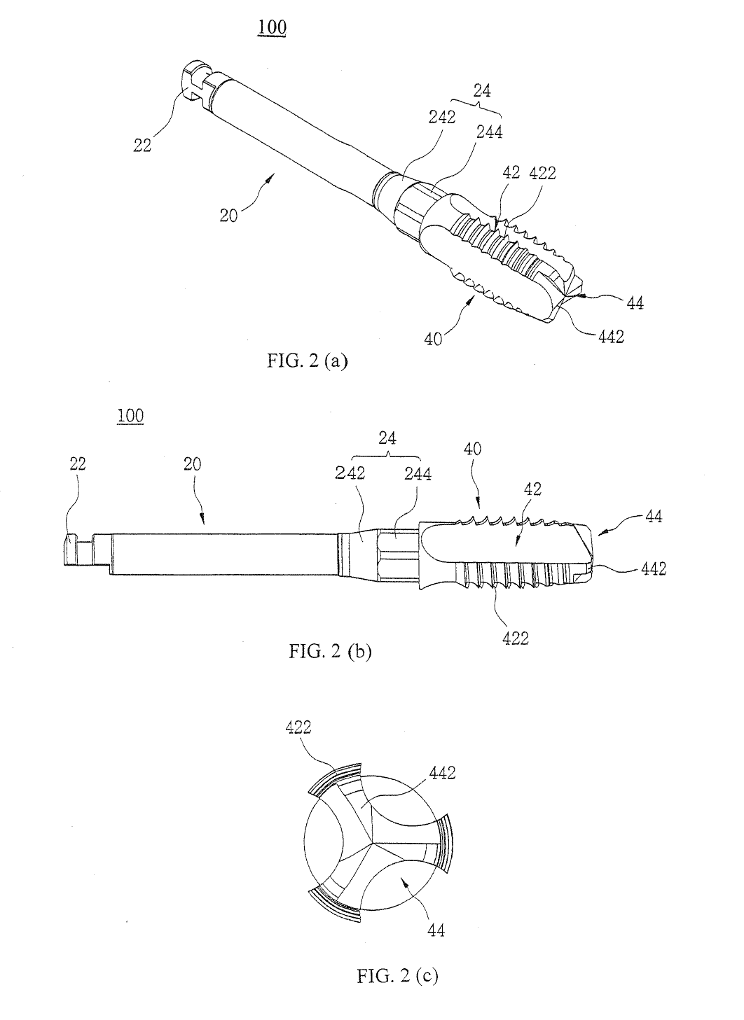

Reference will be now made in detail to the preferred embodiment of the present invention with reference to FIGS. 2 to 7 of the attached drawings. In the drawings and detailed description of the present invention, structures and operations, which can be easily obtained from the general tap drill for the dental implant by those skilled in the art, will be described in brief or omitted. Particularly, while drawings and detailed description of technical structures and operations of parts, which are not directly related with technical characteristics of the present invention, will be omitted, only technical structures related with the present invention will be illustrated in the drawings in brief or described.

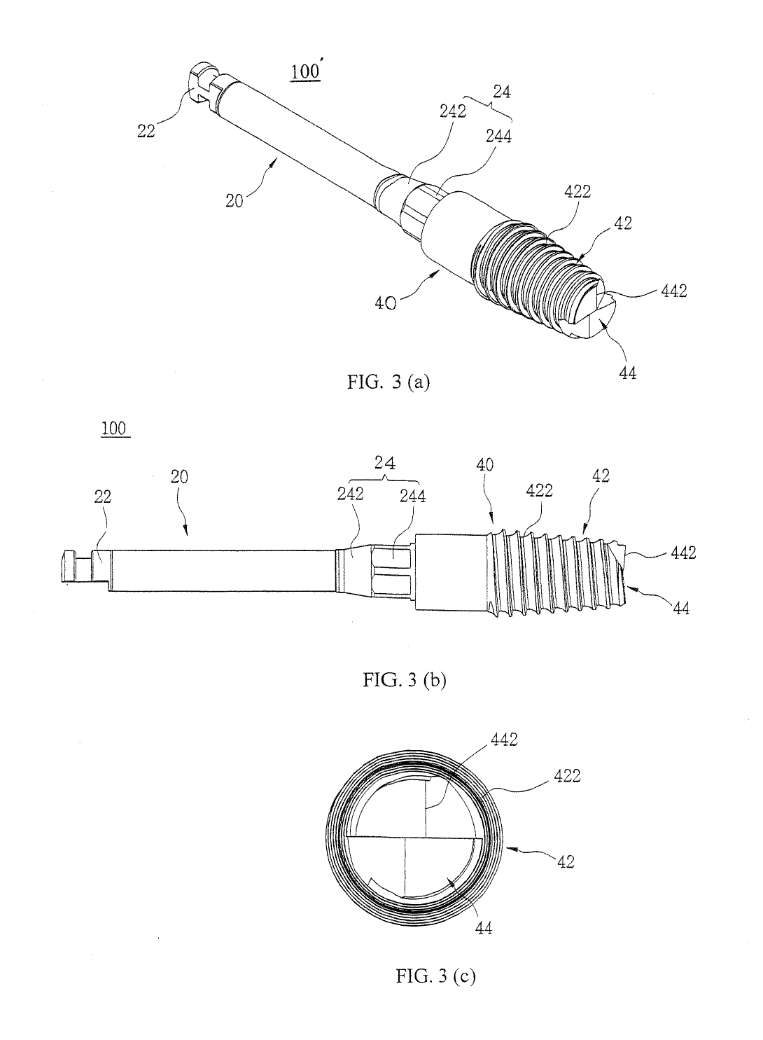

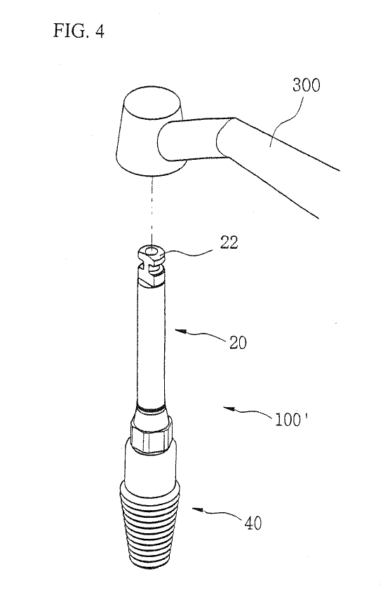

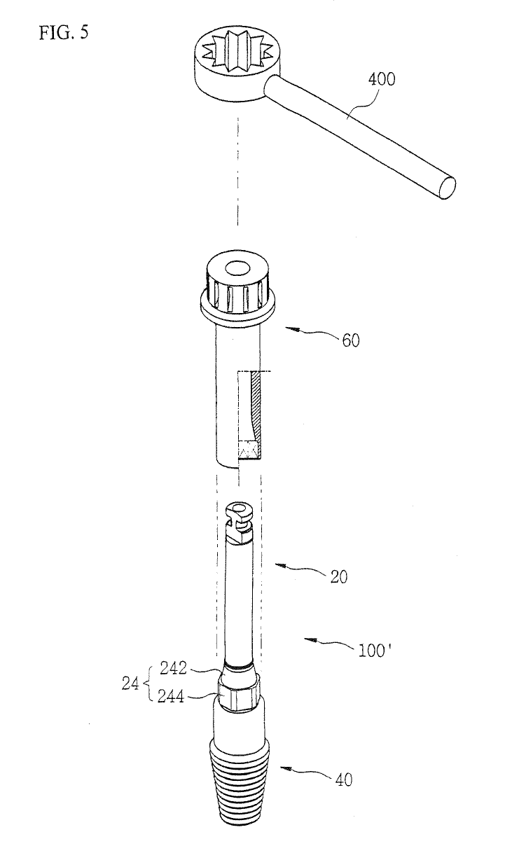

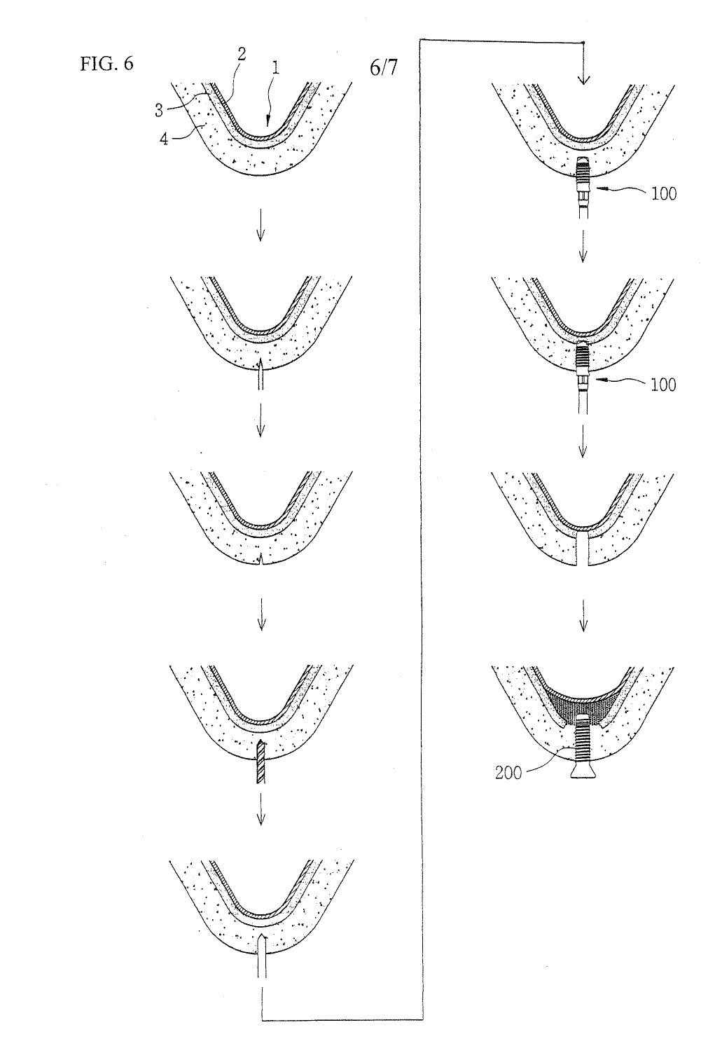

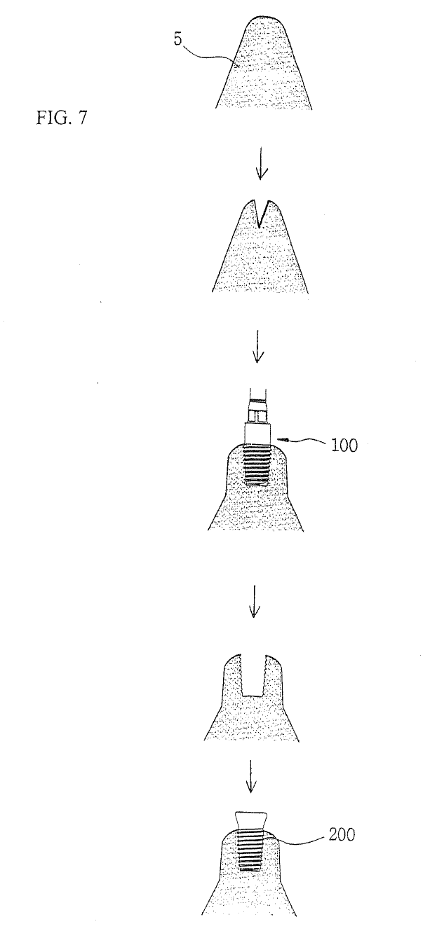

FIG. 2(a) is a perspective view of a straight-type tap drill for a dental implant according to a first preferred embodiment of the present invention, FIG. 2(b) is a sectional view of the straight-type tap drill for the dental implant, FIG. 2(c) is a front view of a cutting blade mounted on the front end face of the straight-type tap drill for the dental implant, FIG. 3(a) is a perspective view of a tapered-type tap drill for a dental implant according to a second preferred embodiment of the present invention, FIG. 3(b) is a sectional view of the tapered-type tap drill for the dental implant, FIG. 3(c) is a front view of a cutting blade mounted on the front end face of the tapered-type tap drill for the dental implant, FIG. 4 is a view showing a state where a handpiece is coupled to the tap drill for the dental implant according to the present invention, FIG. 5 is a view showing a state where a dental ratchet is coupled to the tap drill for the dental implant according to the present invention, FIG. 6 is a view showing a used example of the straight-type tap drill for the dental implant according to the present invention, and FIG. 7 is a view showing a used example of the tapered-type tap drill for the dental implant according to the present invention.

Each of tap drills 100 and 100′ for a dental implant according to the present invention includes: a tapping screw thread 422 formed on the outer peripheral surface of a cylindrical body thereof for forming a space to couple a fixture 200 of the dental implant thereto; and cutting blades 442 radially formed from the center of a front end face 44 of the cylindrical body, so that the tap drill can perform a tapping work of the general tap drill and a cutting work.

As shown in FIGS. 2 and 3, each of the tap drills 100 and 100′ for the dental implant includes a shank part 20 and a tap part 40. The shank part 20 is a part to which a torque transferring mechanism is coupled, and the tap part 40 is a part, which is in direct contact with a cartilaginous tissue or an alveolar bone of a patient's body in order to perform various works for a dental implant surgery. The shank part 20 and the tap part 40 are connected integrally with each other, and so, the tap part 40 is rotated by the torque transferring mechanism coupled to the shank part 20.

For the torque transferring mechanism coupled to the shank part 20, a handpiece 300 as shown in FIG. 4 or a dental ratchet 400 as shown in FIG. 4 may be used. In order to accept both of the handpiece 300 and the dental ratchet 400, each of the tap drills 100 and 100′ for the dental implant according to the present invention has a coupling protrusion 22 adapted to couple the handpiece 300 to the shank part 20 and an adapter mounting portion 24 adapted to couple an adapter 60 of the dental ratchet 400 thereto.

The coupling protrusion 22 is formed at an end of the shank part 20 in such a way as to be directly mounted on the handpiece 300, and the adapter mounting portion 24 is formed at a position spaced apart from the coupling protrusion 22 and coupled to the dental ratchet 400 by means of the adapter 60.

Furthermore, the adapter mounting portion 24 includes a locking face 242 taperedly formed on the outer peripheral surface of the shank part 20, and a contact face 244 formed by the outer peripheral surface of the shank part 20 processed into a polygonal shape. A mounting groove of the adapter 60 of the dental ratchet 400 corresponding to the adapter mounting portion 24 has the same form as the adapter mounting portion 24, so that the adapter 60 of the dental ratchet 400 and the tap drill according to the present invention can be firmly coupled to each other.

In a preferred embodiment of the present invention, the contact face 244 of the adapter mounting portion 24 is formed by the outer peripheral surface of the shank part 20 processed into a hexagonal section form.

As described above, since the tap drill for the dental implant has the coupling protrusion 22 for the handpiece 300 and the adapter mounting portion 24 for the dental ratchet 400, in consideration of a rotational force, rotating speed, precision in work, and so on required for the implant surgery, when any one of the handpiece 300 and the dental ratchet 400 is selected, it can be used for general-purpose without regard to kinds of the torque transferring mechanisms.

The tap part 40 connected to the shank part 20 may be constructed of a straight type as shown in FIGS. 2(a) to 2(c), or a tapered type as shown in FIGS. 3(a) to 3(c).

The tap part 40 includes a tapping screw thread 422 formed on the outer peripheral surface 42 thereof, and cutting blades 442 radially formed from the center of the front end face 44 thereof, which is perpendicular to a direction of a rotary axis when the tap drill rotates.

Here, since the cutting blades 442 formed on the front end face 44 of the tap part 40 have horizontal faces formed at their blade ends, when the tap part 40 is rotated, the cutting blades 442 can crush and face-process matters existing on the front end face 44 of the tap part 40. For a precise cutting, the cutting blades 442 are precisely manufactured through a grinding work. As described above, the cutting blade 442 formed on the tap drill according to the present invention has a structure and an operation similar to an end mill, which is used in a machining work.

FIGS. 2(c) and 3(c) illustrate the cutting blades 442. The cutting blades 442 are symmetric with each other from the center of the front end face 44 of a round form so as to crush the matters existing on the front end face 44 smoothly and stably.

The tap drill having the above configuration according to the present invention is used to bore a fixture insertion hole, to which a fixture 200 of the dental implant can be inserted and fixed from the surface of a cartilaginous tissue or an alveolar bone of a patient's body. Since the tap drill has the cutting blades 442 formed on the front end face 44 of the tap part 40, it can be effectively used in the tapping work against the cartilaginous tissue or the alveolar bone of the patient's body and the cutting work on the cartilaginous tissue or the alveolar bone in the front end direction thereof.

Furthermore, when the tapping work and the cutting work are performed simultaneously against the cartilaginous tissue or the alveolar bone, since the tap part 40 crushes the matters existing on the front end face 44 thereof while rotating at a low speed by means of the torque transferring mechanism, which is used by an operator, in a state where the tap part 40 is laterally supported in stable by the tapping screw thread 422, the tap drill can precisely and stably crush the matters on the front end face 44 to thereby improve precision and stability in the cutting work.

As described above, each of the tap drills 100 and 100′ for the dental implant according to the present invention can be applied to the sinus lift technique as shown in FIG. 6 or the ridge split as shown in FIG. 7 in order to simultaneously perform a boring work to insert and fix the fixture 200 of the dental implant into the alveolar bone and functions required for the sinus lift technique and the ridge split. Such function of the tap drill according to the present invention will be described in detail as follows.

As shown in FIG. 6, in case where the tap drill according to the present invention is applied to the sinus lift technique, first, the fixture insertion hole is formed through the steps of: boring a basic hole in the alveolar bone below a maxillary sinus 1 using a point drill; expanding the basic hole (namely, expanding the basic hole while changing diameters of the drills ranging from a smaller one to a larger one) using general drills for cutting to form the fixture insertion hole; setting the tap drill of the present invention on the fixture insertion hole; and rotating the tap drill using the handpiece 300 or the dental ratchet 400 to thereby process the fixture insertion hole into a female-screwed hole by the tapping screw thread 422 formed on the outer peripheral surface of the tap part 40.

When the tap drill is rotated, the tap drill performs the tapping work while gradually moving in a direction of the maxillary sinus 1, and reaches a compact bone 3 below a maxillary sinus mucosa 2. In the compact bone region, the tap drill is rotated slower to be slowly moved forward, whereby the tap drill can crush and push out the compact bone 3 precisely and stably. Furthermore, since the tapping screw thread 422 formed on the tap part 40 is firmly engaged with the inner peripheral surface of the fixture insertion hole, the tap drill according to the present invention can prevent that the rotary axis is distorted or the tap drill is vibrated when the tap drill is rotated. Therefore, the tap drill according to the present invention can crush the compact bone 3 while being rotated in more stable, and at the same time, moves forward little by little along the tapping screw thread 422 when the tap drill is rotated, so that even operators, who are not highly skilled, can crush only the compact bone 3 without any damage of the maxillary sinus mucosa 2.

Accordingly, if the tap drills 100 and 100′ for the dental implant according to the present invention are applied to the sinus lift technique, the sinus lift technique is enhanced in stability and precision.

Moreover, since the tap drill according to the present invention used for forming the fixture insertion hole can be applied to the sinus lift technique without using the osteotome or the mallet, or the reamer for the dental implant, which is disclosed in Korean Patent No. 10-0,630,304, additionally required in the sinus lift technique, the tap drill according to the present invention can enhance precision and stability in the dental surgery in comparison with the conventional sinus lift technique using the osteotome or the mallet.

As shown in FIG. 7, in case where the tap drill according to the present invention is applied to the ridge split, especially, the tapered-type tap drill 100′ according to the present invention is used. In this instance, first, the fixture insertion hole is formed through the steps of: boring a hole at the center of the narrowed alveolar bone 5; inserting the tap drill 100′ into the hole and rotating the tap drill 100′ using the handpiece 300 or the dental ratchet 400; gradually widening the hole while gradually inserting the tapered-type tap drill 100′ into the hole to form the fixture insertion hole; and inserting and fixing the fixture 200 of the implant into the narrow alveolar bone 5.

While the tap drill for the dental implant according to the present invention has been described with reference to the particular illustrative embodiments, it is not to be restricted by the embodiments but only by the appended claims. It is to be appreciated that those skilled in the art can change or modify the embodiment without departing from the scope and spirit of the present invention.

What is claimed is:

1. A tap drill for a dental implant comprising:
a shank part coupled with a torque transferring mechanism; and
a tap part connected with the shank part, the tap part having: a tapping screw thread formed on the outer peripheral surface thereof for forming a space to insert and fix a fixture of the dental implant thereto; and cutting blades radially formed from the center of a front end face thereof, which is perpendicular to a rotary axis of the tap drill,
whereby the tap drill can simultaneously perform a tapping work, on the outer peripheral surface of the tap part, against a cartilaginous tissue or an alveolar bone of a patient's body, and a cutting work on the front end face of the tap part while the tap part is rotated by the torque transferring mechanism.

2. The tap drill according to claim 1, wherein the tap part adopts one of a straight type and a tapered type.

3. The tap drill according to claim 2, wherein the shank part includes a coupling protrusion formed at an end thereof in such a way as to be coupled to a handpiece and an adapter mounting portion formed at the other end thereof in such a way as to be coupled to an adapter of a dental ratchet, so that the tap part is rotated by one selected from the handpiece and the dental ratchet.

4. The tap drill according to claim 3, wherein the adapter mounting portion includes a locking face taperedly formed on the outer peripheral surface of the shank part, and a contact face formed by processing the outer peripheral surface of the shank part in a polygonal shape.

5. The tap drill according to claim 1, wherein the shank part includes a coupling protrusion formed at an end thereof in such a way as to be coupled to a handpiece and an adapter mounting portion formed at the other end thereof in such a way as to be coupled to an adapter of a dental ratchet, so that the tap part is rotated by one selected from the handpiece and the dental ratchet.

6. The tap drill according to claim 5, wherein the adapter mounting portion includes a locking face taperedly formed on the outer peripheral surface of the shank part, and a contact face formed by processing the outer peripheral surface of the shank part in a polygonal shape.
